(19) United States
(12) Patent Application Publication
(10) Pub. No.: US 2019/0060295 A1
(43) Pub. Date: Feb. 28, 2019

(54) PHARMACEUTICAL COMPOSITION OF A COMBINATION OF TRAMADOL-ETORICOXIB HYDROCHLORIDE FOR THE TREATMENT OF PAIN

(71) Applicant: LABORATORIOS LIOMONT, S.A. DE C.V., Mexico City (MX)

(72) Inventors: Reyna Astrid SALTO RIVERA, Mexico City (MX); Oliver Daniel WALLS FLORES, Mexico City (MX); Ramón RODRÍGUEZ HERNÁNDEZ, Mexico City (MX)

(73) Assignee: Laboratorios Liomont, S.A. DE C.V., Mexxico City (MX)

(21) Appl. No.: 16/008,277

(22) PCT Filed: May 11, 2017
(86) PCT No.: PCT/IB2017/052775

§ 371 Date: Jun. 14, 2018

(30) Foreign Application Priority Data
May 18, 2016 (MX) MX/A/2016/006464

(51) Int. Cl. A61K 31/444; A61K 31/135; A61K 9/20; A61K 9/48; A61K 9/14

(52) U.S. Cl. CPC A61K 31/444; A61K 31/135; A61K 9/14; A61K 9/4833; A61K 9/2095

(57) ABSTRACT

The present disclosure relates to a pharmaceutical composition of a combination of tramadol-etoricoxib hydrochloride for the treatment of pain. The production method of the pharmaceutical composition is based on two active ingredients including granules and/or multi-particulates containing tramadol hydrochloride and granules and/or multi-particulates containing etoricoxib. The pharmaceutical composition can be contained in a mixture of powders, included in a two-layer tablet with or without a coating, or in a capsule as granulates and/or tablets with or without a coating.

### FIELD

The present disclosure consists in an orally administered pharmaceutical composition of a drug containing the tramadol-etoricoxib hydrochloride for the treatment of pain.

### BACKGROUND

There is a need for cyclooxygenase-2 (COX-2) inhibitor to treat pain.

### OBJECT OF THE DISCLOSURE

It includes providing a pharmaceutical composition as a new alternative for the treatment of pain. A further object of the disclosure is to provide an alternative to dosing in a single intake of the two active ingredients etoricoxib-tramadol hydrochloride. Another object of the disclosure is to provide a combination of etoricoxib-tramadol hydrochloride in a pharmaceutical form that may by orally administered.

### DETAILED DESCRIPTION

The active ingredient etoricoxib, is marketed in Mexico as the reference product and with the trade name. Etoricoxib is a highly selective cyclooxygenase-2 (COX-2) inhibitor. The chemical name of etoricoxib is 5-chloro-2-(6-methylpyridin-3-yl)-3-(4-methylsulfonylphenyl)pyridine with an empirical formula C₁₈H₁₅ClN₂O₂S, whose therapeutic indications can be for the acute and chronic treatment of the signs and symptoms of osteoarthritis and rheumatoid arthritis, ankylosing spondylitis, acute gouty arthritis, to relieve acute and chronic pain, primary dysmenorrhea, moderate to severe acute post-surgery pain associated with dental surgery and moderate to severe acute post-surgery pain associated with gynecological abdominal surgery.

The active ingredient tramadol hydrochloride has dual activity as it can demonstrate great agonistic activity on central opioid receptors as well as it inhibiting the re-uptake of norepinephrine and serotonin in the central nervous system, preventing the transmission of pain throughout the bone marrow. The chemical name of tramadol hydrochloride is (1R,2R)-2-(dimethylaminomethyl)-1-(3-methoxyphenyl)cyclohexan-l-ol hydrochloride with an empirical formula C₁₆H₂₆ClNO₂, whose therapeutic indications are for the treatment of moderate pain, intense moderate and neuropathic pain, in the adjuvant treatment of osteoarthritis and in the treatment of restless legs syndrome and post-surgery tremors.

From the search of patents in the drugs gazette of the selective cyclooxygenase-2 inhibitor Etoricoxib, it was found that patent MX 208595 may cover its use as active ingredient until Jul. 8, 2017. From the search in IMPI's database, it was found that patent MX313106 may cover the use of intermediate product for the synthesis of etoricoxib; it was found that patent application MX/a/2017/005316 may cover the manufacturing of a granulate by fluid bed granulation. From the international search, it was found that patent WO 2014/033526 may cover the manufacture of tablets by dry-path to retain the polymorph shape of etoricoxib. In this search, references were found that may cover the topical pharmaceutical form of etoricoxib, of the synthesis and use thereof. Patent WO 2004/093811 may cover the combination of use of a sodium channels blocker with selective cyclooxygenase-2 inhibitors. Patent WO 2004/093813 may cover the combination of use of a calcium blocker modulator with selective cyclooxygenase-2 inhibitors.

The patent of use as active ingredient of the tramadol hydrochloride is of the public domain. In reviewing IMPI's drugs gazette it was found that patent MX266401 may cover the use of the combination of ketorolac with tramadol hydrochloride for the treatment of pain in an oral administration, that patent MX230075 may cover the use of the combination of tramadol hydrochloride and the selection of an anticonvulsant, patent MX275811 which may cover the use of the combination of tramadol hydrochloride with lysine clonixinate to have an analgesic pharmaceutical composition.

This research did not find any combination that discloses selective cyclooxygenase-2-inhibitors as is the special case of etorixocib with tramadol hydrochloride for the treatment of pain.

Based on the above, it is believed that this combination of etoricoxib with tramadol hydrochloride for oral administration is a pharmaceutical product having a high degree of technological innovation as compared to the described backgrounds, such that it qualifies as a beneficial product for Mexican and international societies for the treatment of pain-related allopathy.

For such reasons, a novel pharmaceutical composition of the combination of etoricoxib with tramadol hydrochloride to be orally administered has been developed, which was found to have unexpected effects, as well as the proposed concentration (dosage), which supersedes the effects existing until now. Next, the process of manufacturing is described, which is not limitated but only to exemplify the processes:

1) The surfactant is dispersed in a solvent in a vessel.

2) With a mesh, sieve the etoricoxib or similar salts, one or more diluents, one or more binding agents and one or more disintegrants, add each component to a mixer and mix the powders.

3) With the dissolution of step 1, granulate the mixture of powders of step 2.

4) With a mesh, filter the granulate of step 3 and oven-dry.

5) With a mesh, filter the dry granulate of step 4 and mix with one or more lubricants and/or glidants and/or antiadherents.

6) In a tableting machine, compress the mixture obtained from step 4 to the required concentration between 10 mg and 120 mg of etoricoxib and/or similar salts.

7) In a vessel, disperse at least one tablet coating material in a solvent.

8) In a coating equipment, coat the tablets of step 6 with the dispersion of step 7.

9) In a vessel, disperse at least one binding agent in a solvent.

10) With a mesh, sieve the tramadol hydrochloride or similar salts, one or more diluents, add each component to a mixer and mix the powders.

11) With the dissolution of step 9, granulate the mixture of powders of step 10.

12) With a mesh, filter the granulate of step 11 and oven-dry.

13) With a mesh, filter the dry granulate of step 12 and mix with one or more lubricants and/or glidants and/or antiadherents.

14) In a tableting machine, compress the mixture obtained from step 13 to the required concentration between 10 mg and 30 mg of tramadol hydrochloride and/or similar salts.

15) In a vessel, disperse at least one tablet coating material in a solvent.

16) In a coating equipment, coat the tablets of step 14 with the dispersion of step 15.

17) Fill in capsules with the tablets obtained in steps 8 and 16 to the required dose of etoricoxib or similar salts and tramadol and/or similar salts.

### EXAMPLES

### Example 1

1) The surfactant is dispersed in a solvent in a vessel.

2) With a mesh, sieve the etoricoxib or similar salts, one or more diluents, one or more binding agents and one or more disintegrants, add each component to a mixer and mix the powders.

3) With the dissolution of step 1, granulate the mixture of powders of step 2.

4) With a mesh, filter the granulate of step 3 and oven-dry.

5) With a mesh, filter the dry granulate of step 4 and mix with one or more lubricants and/or glidants and/or antiadherents.

6) In a vessel, disperse at least one solvent in a binding agent.

7) With a mesh, sieve the tramadol hydrochloride or similar salts, one or more diluents, add each component to a mixer and mix the powders.

8) With the dissolution of step 6, granulate the mixture of powders of step 7.

9) With a mesh, filter the granulate of step 8 and oven-dry.

10) With a mesh, filter the dry granulate of step 9 and mix with one or more lubricants and/or glidants and/or antiadherents.

11) In a tableting machine, compress the mixture obtained from steps 5 and 10 to the required concentration between 10 mg and 120 mg of etoricoxib and/or similar salts and between 10 mg and 300 mg of tramadol hydrochloride and/or similar salts.

### Example 2

1) The surfactant is dispersed in a solvent in a vessel.

2) With a mesh, sieve the etoricoxib or similar salts, one or more diluents, one or more binding agents and one or more disintegrants, add each component to a mixer and mix the powders.

3) With the dissolution of step 1, granulate the mixture of powders of step 2.

4) With a mesh, filter the granulate of step 3 and oven-dry.

5) With a mesh, filter the dry granulate of step 4 and mix with one or more lubricants and/or glidants and/or antiadherents.

6) In a vessel, disperse at least one binding agent in a solvent.

7) With a mesh, sieve the tramadol hydrochloride or similar salts, one or more diluents, add each component to a mixer and mix the powders.

8) With the dissolution of step 6, granulate the mixture of powders of step 7.

9) With a mesh, filter the granulate of step 8 and oven-dry.

10) With a mesh, filter the dry granulate of step 9 and mix with one or more lubricants and/or glidants and/or antiadherents.

11) In a tableting machine, compress the mixture obtained from step 10 to the required concentration between 10 mg and 300 mg of tramadol hydrochloride and/or similar salts.

12) Fill in capsules with the mixture of powders obtained in step 5 and with the tablets obtained in step 11 to the required dose of 10 mg to 120 mg of etoricoxib and/or similar salts and from 10 mg to 300 mg of tramadol hydrochloride and/or similar salts.

### Example 3

1) The surfactant is dispersed in a solvent in a vessel.

2) With a mesh, sieve the etoricoxib or similar salts, one or more diluents, one or more binding agents and one or more disintegrants, add each component to a mixer and mix the powders.

3) With the dissolution of step 1, granulate the mixture of powders of step 2.

4) With a mesh, filter the granulate of step 3 and oven-dry.

5) With a mesh, filter the dry granulate of step 4 and mix with one or more lubricants and/or glidants and/or antiadherents.

6) In a vessel, disperse at least one binding agent in a solvent.

7) With a mesh, sieve the tramadol hydrochloride or similar salts, one or more diluents, add each component to a mixer and mix the powders.

8) With the dissolution of step 6, granulate the mixture of powders of step 7.

9) With a mesh, filter the granulate of step 8 and oven-dry.

10) With a mesh, filter the dry granulate of step 9 and mix with one or more lubricants and/or glidants and/or antiadherents.

11) Fill in capsules with the mixture of powders obtained in step 5 and step 10 to the required dose of 10 mg to 120 mg of etoricoxib and/or similar salts and from 10 mg to 300 mg of tramadol and/or similar salts.

1-6. (canceled).

7. A pharmaceutical composition for the oral treatment of pain comprising:
a combination of tramadol hydrochloride or a pharmaceutically acceptable salt thereof with etoricoxib or a pharmaceutically acceptable salt thereof.

8. The pharmaceutical composition of claim 7, wherein a particle size of etorixocib or a pharmaceutically acceptable salt thereof is less than or equal to 30 microns.

9. The pharmaceutical composition of claim 7, containing granules or particulate material containing etoricoxib or a pharmaceutically acceptable salt thereof and/or tramadol hydrochloride or a pharmaceutically acceptable salt thereof individually or combined which allows for the oral co-administration thereof.

10. The pharmaceutical composition of claim 7, containing a dosage of 10 to 120 mg of etoricoxib or a pharmaceutically acceptable salt thereof with
tramadol hydrochloride or a pharmaceutically acceptable salt thereof in a dosage of 10 to 300 mg.

11. The pharmaceutical composition of claim 7, containing a dosage of 10 to 120 mg of etoricoxib or a pharmaceutically acceptable salt thereof with
tramadol hydrochloride or a pharmaceutically acceptable salt thereof in a dosage of 10 to 300 mg for the treatment of pain.

12. A method of orally administering a pharmaceutical composition for the treatment of pain, the pharmaceutical composition comprising:
a combination of tramadol hydrochloride or a pharmaceutically acceptable salt thereof with etoricoxib or a pharmaceutically acceptable salt thereof.

13. The method of claim 12, wherein a particle size of etorixocib or a pharmaceutically acceptable salt thereof is less than or equal to 30 microns.

14. The method of claim 12, wherein the pharmaceutical composition contains granules or particulate material containing etoricoxib or a pharmaceutically acceptable salt thereof and/or
tramadol hydrochloride or a pharmaceutically acceptable salt thereof individually or combined which allows for the oral co-administration thereof.

15. The method of claim 12, wherein the pharmaceutical composition contains a dosage of 10 to 120 mg of etoricoxib or a pharmaceutically acceptable salt thereof with
tramadol hydrochloride or a pharmaceutically acceptable salt thereof in a dosage of 10 to 300 mg for the treatment of pain.

16. The method of claim 12, wherein the pharmaceutical composition of etoricoxib or a pharmaceutically acceptable salt thereof in combination with tramadol hydrochloride or a pharmaceutically acceptable salt thereof is administered individually or separately, at the same time or interchangeably one before the other, to suppress the effects of pain.
